(19) United States
(12) Patent Application Publication
(10) Pub. No.: US 2024/0165557 A1
(43) Pub. Date: May 23, 2024

(54) METHODS OF ADDING FEED MEDIUM INTO BIOPROCESS

(71) Applicant: Solar Foods Oy, Lappeenranta (FI)

(72) Inventor: Petri TERVASMÄKI, Helsinki (FI)

(73) Assignee: Solar Foods Oy, Lappeenranta (FI)

(21) Appl. No.: 18/550,850

(22) PCT Filed: Apr. 26, 2022
(86) PCT No.: PCT/FI2022/050271

§ 371 Date: Sep. 15, 2023

(30) Foreign Application Priority Data
Apr. 27, 2021 (FI) 20215486

(51) Int. Cl. B01D 53/62; B01D 53/14; B01D 53/18; B01D 53/73; B01D 53/78; C12M 1/00; C12M 1/34

(52) U.S. Cl. CPC B01D 53/62; B01D 53/145; B01D 53/1475; B01D 53/18; B01D 53/73; B01D 53/78; C12M 29/24; C12M 41/34; C12M 43/04; B01D 2252/103; B01D 2257/504; B01D 2258/0283

(57) ABSTRACT

Disclosed is a method of adding a feed medium into a bioprocess. The method includes receiving a stream of CO2-rich gas; treating the stream of CO2-rich gas to remove impurities therefrom; preparing an aqueous mixture for absorbing carbon dioxide, the aqueous mixture having at least one inorganic nitrogen compound in a range of 0.1-50 wt % of the aqueous mixture, the at least one inorganic nitrogen compound is a nitrogen source for microorganisms; absorbing carbon dioxide from the stream of CO2-rich gas into the aqueous mixture, the aqueous mixture with absorbed carbon dioxide forming a feed medium; and adding the feed medium into a bioprocess.

### TECHNICAL FIELD

The present disclosure relates generally to carbon dioxide capturing process; and more specifically to methods and systems of adding feed medium into bioprocess.

### BACKGROUND

Carbon dioxide (CO₂) is a greenhouse gas that absorbs and radiates heat and leads to global warming. Global average atmospheric CO₂ level has raised concerns in an alarming way. The atmospheric CO₂ level is rising due to natural processes (such as volcanic eruptions), burning of fossil fuels (such as coal and oil), and CO₂ emissions (such as chlorofluorocarbons) as a result of various industrial activities. In this regard, governmental organizations, world-wide, have laid restrictions on industries for releasing reduced amounts of CO₂ in atmosphere, and encourage CO₂ recycling therefor.

Generally, CO₂ recycling involves capturing carbon dioxide emitted from one process, such as for example industrial side streams (e.g., flue gases), and having a separate CO₂ capture process and adding the gaseous CO₂ into another process, such as for example a bioprocess. Specifically, adding CO₂ into a bioprocess (such as microbe cultivation) as a carbon source therein requires relatively high amount of gaseous CO₂ as an input to a bioreactor containing an aqueous phase growth medium.

Moreover, the conventional CO₂ recycling techniques introduce complexity in terms of energy requirements and several process stages with dedicated equipment. For example, capturing CO₂ from industrial side streams requires energy, and several process stages, such as compression, decompression, absorption, desorption, and regeneration of purified CO₂ gas for feeding to a bioprocess. Moreover, besides absorption of CO₂ from the CO₂-rich gas stream into a solvent liquid (most commonly water, amines, salt solutions, aqueous ammonia) and desorption of CO₂ as purified gas, the integration process further requires an additional step of mixing with a growth medium (namely, bioprocess feed) during integration into the bioprocess. Thereby, making the integration process energy-inefficient and time-consuming.

Therefore, in light of the foregoing discussion, there exists a need to overcome drawbacks associated with conventional techniques of integrating CO₂ from external processes into the bioprocess.

### SUMMARY

The present disclosure seeks to provide a method of adding a feed medium into a bioprocess. The present disclosure also seeks to provide a system for adding a feed medium into a bioprocess. The present disclosure seeks to provide a solution to the existing problem of carbon dioxide (CO₂) capturing process and its integration to a bioprocess. An aim of the present disclosure is to provide a solution that overcomes at least partially the problems encountered in prior art.

In an aspect, an embodiment of the present disclosure provides a method of adding a feed medium into a bioprocess, the method comprising:
(a) receiving a stream of CO₂-rich gas;
(b) treating the stream of CO₂-rich gas to remove impurities therefrom;
(c) preparing an aqueous mixture for absorbing carbon dioxide, the aqueous mixture comprises at least one inorganic nitrogen compound in a range of 0.1-50 wt % of the aqueous mixture, the at least one inorganic nitrogen compound is a nitrogen source for microorganisms, and absorbing carbon dioxide from the stream of CO₂-rich gas into the aqueous mixture, the aqueous mixture with absorbed carbon dioxide forming a feed medium; and
(d) adding the feed medium into a bioprocess.

In another aspect, an embodiment of the present disclosure provides a system of adding a feed medium into a bioprocess, the system comprising:
a first inlet for providing a stream of CO₂-rich gas;
a pre-filter for treating the stream of CO₂-rich gas to remove impurities therefrom;
an absorption chamber for absorbing carbon dioxide from the stream of CO₂-rich gas, and a second inlet for receiving an aqueous mixture that absorbs the carbon dioxide to form a feed medium, the aqueous mixture comprises at least one inorganic nitrogen compound in a range of 0.1-50 wt % of the aqueous mixture, the at least one inorganic nitrogen compound is a nitrogen source for microorganisms;
a third inlet for adding the feed medium into a bioprocess; and
a bioreactor for facilitating the bioprocess.

Embodiments of the present disclosure substantially eliminate or at least partially address the aforementioned problems in the prior art, and provides an efficient method of capturing CO₂ from external sources and dissolution thereof with the bioprocess feed. Beneficially, the disclosed method eliminates multiple process steps (such as for example CO₂ absorption step, CO₂ desorption step, storage of gaseous CO₂, and dissolution of CO₂), thereby requiring less equipment for the entire process.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a flowchart depicting steps of a method of adding a feed medium into a bioprocess, in accordance with an embodiment of the present disclosure; and

FIGS. 2 and 3 are schematic illustrations of a system for adding a feed medium into a bioprocess, in accordance with different embodiments of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

In an aspect, an embodiment of the present disclosure provides a method of adding a feed medium into a bioprocess, the method comprising: (a) receiving a stream of CO₂-rich gas;
(b) treating the stream of CO₂-rich gas to remove impurities therefrom;
(c) preparing an aqueous mixture for absorbing carbon dioxide, the aqueous mixture comprises at least one inorganic nitrogen compound in a range of 0.1-50 wt % of the aqueous mixture, the at least one inorganic nitrogen compound is a nitrogen source for microorganisms, and absorbing carbon dioxide from the stream of CO₂-rich gas into the aqueous mixture, the aqueous mixture with absorbed carbon dioxide forming a feed medium; and
(d) adding the feed medium into a bioprocess.

In another aspect, an embodiment of the present disclosure provides a system of adding a feed medium into a bioprocess, the system comprising:
a first inlet for providing a stream of CO₂-rich gas;
a pre-filter for treating the stream of CO₂-rich gas to remove impurities therefrom;
an absorption chamber for absorbing carbon dioxide from the stream of CO₂-rich gas, and a second inlet for receiving an aqueous mixture that absorbs the carbon dioxide to form a feed medium, the aqueous mixture comprises at least one inorganic nitrogen compound in a range of 0.1-50 wt % of the aqueous mixture, the at least one inorganic nitrogen compound is a nitrogen source for microorganisms;
a third inlet for adding the feed medium into a bioprocess; and
a bioreactor for facilitating the bioprocess.

The present disclosure provides the aforementioned method and system for adding the feed medium into the bioprocess. The method of the present disclosure utilizes feed flow from external sources as an input to absorb CO₂ gas therefrom and feed the absorbed CO₂ gas as a part of the bioprocess feed. Beneficially, such integration of the CO₂ capturing process and the bioprocess saves energy and cost that would be required for gas compression and dissolution in the CO₂ capture process before feeding the CO₂ gas to the bioprocess. Additionally, beneficially, lesser number of intermediate steps reduces the number of dedicated equipments, thereby, ensuring easy and safe handling of the CO₂ gas as well as the end product resulting from the bioprocess.

The present disclosure provides a method and system of adding a feed medium into a bioprocess. Herein, the term “bioprocess” refers to a process that employs living cells or their components (for example, microorganisms, enzymes and the like) to obtain intended products from the bioprocess. The bioprocess may involve culturing cells, growing micro-organisms, production of biomolecules and so forth. The system comprises a bioreactor for facilitating the bioprocess. Herein, the term “bioreactor” refers to a vessel intended to support and facilitate bioprocess therein. Furthermore, volume of the bioreactor is selected depending upon its use. The bioreactor may be fabricated of a material that is inert to the contents of the bioreactor. In an example, the material used for fabrication may be stainless steel (for example type 304, 316 or 316L), other suitable metals or alloys, glass material, fibres, ceramic, plastic materials and/or combinations thereof. Moreover, the fabrication material is typically waterproof and strong enough to withstand abrasive effects of various biological, biochemical and/or mechanical processes, such as microorganism concentrations, biomass productions, agitation forces, aeration forces, operating pressures, temperatures and so forth.

Optionally, the bioreactor is configured for cultivating microorganisms. Microorganisms require suitable environmental conditions such as temperature, pressure and pH, and the bioreactor is equipped with means of controlling the environmental conditions. Optionally, the microorganisms are selected from a group comprising autotrophic microorganisms, heterotrophic microorganisms or mixotrophic organisms. Optionally, the bioreactor is configured for cultivating microorganisms selected from a group comprising aerobic microorganisms, anaerobic microorganisms or facultative anaerobic microorganisms. Notably, autotrophic microorganisms can use carbon dioxide as their carbon source to convert to organic carbon compounds. Furthermore, autotrophic microorganisms acquire their energy from light or from chemical compounds (chemotrophs) to generate organic compounds. Heterotrophic refers to microorganisms that utilize organic carbon as carbon sources. Mixotrophic refers to microorganisms that can function both autotrophically and heterotrophically. Moreover, many bioprocesses, such as gas fermentation process, involve use of certain types of bacteria that utilize chemical energy to convert CO₂ into different organic compounds. The facultative anaerobic microorganisms refer to microorganisms that can function in aerobic, anoxic, or anaerobic conditions and are employed in a variety of bioprocesses. In this regard, the facultative anaerobic microorganisms make adenosine-triphosphate by aerobic respiration if oxygen is present but is capable of switching to fermentation or anaerobic respiration if oxygen is absent.

The method comprises receiving a stream of CO₂-rich gas. The system comprises a first inlet for providing a stream of CO₂-rich gas. Notably, the stream of CO₂-rich gas has higher concentration of CO₂ than 400 parts per million i.e., higher than concentration of CO₂ in atmosphere. Specifically, the stream of CO₂-rich gas may have a concentration of CO₂ higher than 30 percent of the total volume of the stream of CO₂-rich gas. In an implementation, the stream of CO₂-rich gas may be a side stream or obtained as a by-product from industrial process.

In an embodiment, the CO₂-rich gas is obtained from an external source, and wherein the external source is a combustion plant. Optionally, the organic compounds used as a fuel for combustion plant includes both fossil resources and renewable resources like wood. It will be appreciated that combustion of organic compounds is a potential source of CO2-rich gas. Optionally, the combustion plant is selected from at least one of: an electric power facility, central heating facility, other coal-based facility. Notably, electric power facility, for example a coal-fired power plant, and other combustion plants generally, generate large amounts of CO₂-rich gas as a result of burning of coal. Similarly, central heating facilities produce the stream of CO₂-rich gas as they employ fossil fuels for operation.

Moreover, CO2-rich gas may be obtained from other potential routes such as microbial processing of organic compounds. The external source may comprise anaerobic digestion chambers, ethanol production facilities, bioethanol production facilities for microbial fermentation processes. The microbial fermentation processes contain higher CO₂-concentration compared to e.g. common power plants allowing higher CO₂ absorption capacity and making the CO₂ absorption process more efficient and faster. Optionally, the CO₂-rich gas is obtained from processing of carbonate containing minerals, for example limestone calcination.

Optionally, the stream of CO₂-rich gas may comprise a recycled gas stream, which comprises at least one of selected from the carbon dioxide, water and one or more plurality of insoluble gases generated at absorbing carbon dioxide from the stream of CO₂-rich gas or the carbon dioxide generated in the bioprocess.

It will be appreciated, that the microbial fermentation process could be the aforementioned bioprocess (or the bioreactor). Notably, the bioprocess utilizes supplied CO₂ and release some amount of unutilized CO₂ as the by-product of the bioprocess. Such CO₂ released as the by-product could be recycled back as a source of CO₂-rich gas for an efficient utilization of the CO₂ in the integrated CO₂ capturing process.

The method comprises treating the stream of CO₂-rich gas to remove impurities therefrom. The treating comprises filtering the stream of CO₂-rich gas and optionally a treatment method selected based on the impurities to be removed. The system comprises a pre-filter for treating the stream of CO₂-rich gas to remove impurities therefrom. Notably, impurities refer to any undesirable chemical compounds in the CO₂-rich gas. If not removed, the impurities may initiate an undesirable reaction when the CO₂-rich gas is absorbed in the aqueous mixture. Moreover, the impurities may also initiate undesirable reactions in the bioprocess wherein for example sulphurous gases may have an adverse effect on the growth of microorganisms.

Moreover, treating the CO₂-rich gas comprises at least one of selected from filtering; pre-scrubbing; using a flash tank; desulphurisation; removal of hydrocarbons, oxygen, halogen, siloxanes; filtering as high-efficiency particulate absorbing filtering. The system may further comprise at least one of selected from a pre-scrubber, a flash tank, an adsorber, a micro-aerator, a high-efficiency particulate absorbing filter. In addition to filtering the stream of CO₂-rich gas with a pre-filter, the stream of CO₂-rich gas may be treated with a selected treating method. The pre-filter or the treating method employed thereby is selected based on the impurities that are known to be present in the stream of CO₂-rich gas or may be selected based on the source of the stream of CO₂-gas. The treating method is selected from at least one of: desulphurisation i.e., removal of sulphurous gases (via adsorption or in-situ micro-aeration), removal of hydrocarbons, oxygen, halogen, siloxanes. The pre-filter is selected based on the treating method employed for removing the impurities.

Moreover, particulate impurities are required to be removed before the CO₂ absorption stage. It will be appreciated that the amount and type of particulate impurities could affect the filtering stage and higher particulate impurity concentration could increase the pressure drop during the filtering stage, thereby, resulting in an increased energy demand for gas compression. Furthermore, gaseous impurities could be removed either before or after the absorption stage. In addition to treating the stream of CO₂-rich gas with filtering and/or selected treating method a pre-scrubber may be used to remove the particulate impurities before the absorption stage. Furthermore, a flash tank may be used to remove to remove other, less soluble gases, for example nitrogen (N₂). It will be appreciated that the amount and concentration of gaseous impurities determine whether the pre-scrubber (before absorption stage) or the flash tank (after absorption stage) are required, and based thereon, the design parameters of pre-scrubber or flash tank are determined.

Treating the stream of CO₂-rich gas generally comprises filtering with the pre-filter and may be optionally complemented with another selected treating method as mentioned previously. Furthermore, to remove particulate impurities from the stream of CO₂-rich gas, a pre-scrubber may be used before the absorption stage in addition to filtering and selected treating method.

Optionally, treating the stream of CO₂-rich gas comprises filtering as high-efficiency particulate absorbing filtering (HEPA filtering). Notably, the CO₂-rich gas undergoes HEPA filtering to remove any impurities below a given diameter, for example 0.3 micrometre (μm), from the CO₂-rich gas. Furthermore, HEPA filtering removes at least 99.97% of dust, pollen, mold, bacteria, and any airborne particles from the CO₂-rich gas that may cause unintended effects (such as toxic, pathogenic, fungal growth, and so forth) in the absorption chamber.

The method comprises preparing the aqueous mixture for absorbing carbon dioxide, the aqueous mixture comprises at least one inorganic nitrogen compound in a range of 0.1-50 wt % of the aqueous mixture, the at least one inorganic nitrogen compound is a nitrogen source for microorganisms. The at least one inorganic nitrogen compound may be selected from aqueous solution of amines, ammonia, or inorganic nitrogen salts. Notably, amines, ammonia or inorganic nitrogen salts may increase solubility of the aqueous mixture with respect to carbon dioxide, thereby allowing a higher quantity of carbon dioxide to be absorbed therein. In an example, the aqueous mixture comprises an aqueous solution of ammonia that upon absorbing carbon dioxide forms ammonium bicarbonate, i.e.,

CO₂(g)+NH₃(aq.)+H₂O→(NH₄)HCO₃(aq.)

Notably, inorganic nitrogen salts in the feed medium may form a nitrogen source for the microorganisms in the bioprocess. Herein, the absorption of carbon dioxide in the aqueous mixture enables separation of carbon dioxide from other gases present in the CO₂-rich gas. Increased concentration of inorganic nitrogen compound increases the amount of CO₂ that can be captured via the present method. However, some inorganic nitrogen compounds, like for example ammonium bicarbonate, may precipitate more easily in higher concentration. Therefore, there is a need for optimal range of the inorganic nitrogen compound in the aqueous mixture. The aqueous solution may for example comprise at least one inorganic nitrogen compound from 0.1, 0.2, 0.3, 0.4, 0.5, 1, 2, 3, 4, 5, 7.5, 10, 15, 20, 25, 30, 35, 40, 45 weight percent (wt %) up to 0.5, 1, 2, 3, 4, 5, 7.5, 10, 15, 20, 25, 30, 35, 40, 45, 50 weight percent of the aqueous mixture. Beneficially, the aqueous mixture is an efficient form of physical absorption of carbon dioxide. Additionally, beneficially, employing aqueous mixture eliminates the need for heating or steam generation during the carbon dioxide absorption process. Moreover, addition of suitable solvents in the aqueous mixture enhances the absorption of carbon dioxide, such as by using carbon dioxide as one of the reactants.

In an embodiment, the concentration of the at least one inorganic nitrogen compound is in a range of 5-10 wt % of the aqueous mixture. For example, if the concentration of the aqueous nitrogen, for example aqueous ammonia, is more than 15 wt % of the aqueous mixture, a lot of nitrogen, including for example ammonia, will volatilize from the solution, furthermore, low concentration of nitrogen can also have higher rates of removal according to experiments. However, inorganic nitrogen salts in the feed medium will form a nitrogen source for the microorganisms in the bioprocess. Therefore, the optimal concentration of inorganic nitrogen compound is selected to be from 5 wt %, 6 wt %, 7 wt %, 8 wt % up to 8 wt %, 9 wt %, 10 wt %.

The method comprises absorbing carbon dioxide from the stream of CO₂-rich gas into the aqueous mixture, the aqueous mixture with absorbed carbon dioxide forming a feed medium. The system comprises an absorption chamber for absorbing carbon dioxide from the stream of CO₂-rich gas. The absorbed carbon dioxide is coupled with a second inlet for receiving an aqueous mixture that mixes with the absorbed carbon dioxide to form feed medium. Herein, the absorption chamber is an industrial equipment used to separate gases by absorption (or scrubbing) with a suitable liquid. Examples of the absorption chamber include, but are not limited to, a packed column, a plate tower, a simple spray column, a bubble column, or an in-line equipment such as ejector-venturi scrubber. Notably, absorption of CO₂-rich gas in the aqueous medium allows phase change of the carbon dioxide in the CO₂-rich gas and separation from other gases present therein. The absorbed carbon dioxide mixed with the aqueous mixture forms feed medium for microorganisms in the bioprocess. As mentioned previously, the microorganisms in the bioprocess use carbon dioxide as a carbon source to convert in to organic carbon compounds. Beneficially, absorbing the carbon dioxide from the stream of CO₂-rich gas in the aqueous mixture allows the feed medium to be added directly to the bioprocess without a separate regeneration and CO₂ capture process, thereby reducing complexity and cost of the process. Furthermore, adding carbon dioxide as a feed medium reduces gaseous inputs to the bioreactor.

Optionally, the absorption of carbon dioxide is carried out at temperature ranging from 0 to 35° C. and pressure ranging from 1 to 200 bars. Notably, the temperature and pressure range enable optimum dissolution of carbon dioxide in the aqueous mixture. In an example, the aqueous mixture comprises an aqueous solution of ammonia. In such example, temperature ranging from 25 to 35° C. and pressure ranging from 1 to 10 bar avoids precipitation and decomposition of ammonium bicarbonate (in the absorption chamber or with in-line absorber) and maximises dissolution of carbon dioxide in the aqueous mixture. The absorption of carbon dioxide may, for example be carried out at temperatures from 0, 5, 10, 15, 20, 25, 30 degrees Celsius up to 5, 10, 15, 20, 25, 30, 35 degrees Celsius. The absorption of carbon dioxide may, for example be carried out at pressure from 1, 5, 10, 15, 20, 40, 60, 80, 100, 120, 140, 160 or 180, bars up to 5, 10, 15, 20, 40, 60, 80, 100, 120, 140, 160, 180 or 200 bars.

Optionally, the method further comprises filtering the feed medium to remove impurities selected from plurality of solid impurities. Optionally, the system comprises a filter for filtering the feed medium by removing impurities selected from plurality of solid impurities. Notably, the impurities are removed to ensure that they do not enter the bioreactor and affect the bioprocess in any unintended manner. The filter removes any impurities caused by decomposition or precipitation of solvents in the aqueous mixture. In an example, the filter removes any precipitated ammonium bicarbonate from the feed medium, in an event the aqueous solution of ammonia is used in the aqueous mixture.

Optionally, filtering is sterile filtering. Optionally, the filter is a sterile filter. Notably, the feed medium undergoes sterile filtering to remove any impurities below a given diameter, for example 0.2 micrometre (μm), from the feed medium. Furthermore, sterile filtering removes contaminating microorganisms from the feed medium that may cause unintended effects (such as toxic, pathogenic, fungal growth, and so forth) in the bioprocess when added to the bioreactor.

Optionally, the method comprises further recycling a recycled gas stream back to receiving the stream of CO₂-rich gas. The recycled gas stream comprises at least one of selected from the carbon dioxide, water and one or more plurality of insoluble gases generated at absorbing carbon dioxide from the stream of CO₂-rich gas or the carbon dioxide generated in the bioprocess. The stream of CO₂-rich gas is supplemented by the recycled gas stream. The system further comprises at least one recycle unit. Optionally, the system may comprise at least one sensor element configured to measure at least one of selected from the concentration of carbon dioxide, water and one or more plurality of insoluble gases generated in the adsorption chamber or the carbon dioxide generated in the bioreactor for determining the concentration of CO₂ necessary at the first inlet. The at least one recycle unit may be communicably coupled to the absorption chamber and the filter, configured to recycle the carbon dioxide, water and one or more plurality of insoluble gases. Herein, the recycled carbon dioxide could not be absorbed in the aqueous mixture and therefore, is recycled to the absorption chamber for resorption. The insoluble gases may include, but are not limited to, nitrogen, methane, carbon dioxide. The recycle unit removes such insoluble gases and water vapour and recirculates to the absorption chamber. Beneficially, the recirculation of the insoluble gases enables efficient absorption of trace amounts of carbon dioxide that were not absorbed earlier in the water column. Additionally, beneficially, recirculation of water vapour enables maintaining the water column and eliminates the need of energy-extensive, continuous supply of purified water for carbon dioxide absorption in the absorption chamber. The recycle unit or a flash tank may have a reduced pressure in comparison with the absorption chamber to allow escape of the carbon dioxide, water and one or more plurality of insoluble gases from the feed medium. In an example, the pressure of the recycle unit may be in a range of 25 to 75 percent of the pressure of the absorption chamber. The at least one recycle unit may be further communicably coupled to the bioreactor for recycling back CO₂ content generated therein during the bioprocess as a by-product.

The CO₂ concentration of the total volume of the stream of CO₂-rich gas supplemented by the recycled gas stream is determined by equation:

$$\\ X = \frac{( A \times B + C \times D )}{B + C} ,$$

where

X is concentration of CO₂ in the total volume of the stream of CO₂-rich gas supplemented by the recycled gas stream,

A is concentration of CO₂ in the stream of CO₂-rich gas,

B is flow rate of the stream of CO₂-rich gas,

C is flow rate of the recycled gas stream, and

D is concentration of CO₂ in the recycled gas stream.

The concentration of CO₂ at receiving the stream of CO₂-rich gas supplemented by the recycled gas stream is dependent of the concentration of CO₂ in the stream of CO₂-rich gas and flow rates of the stream of CO₂-rich gas and recycled gas stream. This way an optimal concentration of CO₂ can be obtained at receiving a stream of CO₂-rich gas supplemented by the recycled gas stream at a first inlet. The stream of CO₂-rich gas that is received from an external source will be supplemented with the recycled gas stream.

The method comprises adding the feed medium into a bioprocess. The system comprises a third inlet for adding the feed medium into a bioprocess. The feed medium comprising absorbed carbon dioxide provides a carbon source for the microorganisms in the bioreactor. The feed medium provides a liquid medium for the bioprocess comprising absorbed carbon dioxide and water. The bioreactor facilitates a continuous bioprocess with agitation to ensure uniform mixing of the feed medium with contents of the bioreactor. Furthermore, the pH of the feed medium is controlled in a manner that allows growth of microorganisms in the bioreactor. In an embodiment, the feed medium further comprises ammonium bicarbonate that provides a nitrogen source for the microorganisms.

Optionally, the method further comprises adding at least one of: hydrogen gas, oxygen gas, carbon monoxide, minerals, light into the bioprocess. The system further comprises at least one fourth inlet for adding at least one of: hydrogen gas, oxygen gas, carbon monoxide, minerals into the bioprocess; and a light source coupled to the bioreactor for illumination thereof. Notably, addition of hydrogen gas, oxygen gas, carbon monoxide, minerals, light into the bioprocess is performed based on the type of bioprocess and the microorganisms involved therein. For example, hydrogen gas is generally used as an energy source for autotrophic microorganisms and may be used in processes such as gas fermentation (namely, syngas fermentation). Notably, syngas fermentation is an anaerobic process wherein introduction of oxygen has to be avoided for production of ethanol or other commodity chemicals. Furthermore, carbon monoxide may be added as an additional carbon and energy source in bioprocesses such as syngas fermentation. For bioprocesses such as gas fermentation using aerobic microorganisms, carbon monoxide, hydrogen gas and oxygen gas may be added for growth of autotrophic microorganisms such as hydrogen-oxidising bacteria. For bioprocesses involving heterotrophic microorganisms, phototrophic microorganisms or facultative anaerobic microorganisms, the light from the light source further facilitates the bioprocess wherein wavelength of a photosynthetically active radiation (PAR) is considered to be between 400 and 700 nm. Additionally, nutrients and minerals are added to the bioreactor to aid growth and functioning of microorganism.

It will be appreciated that the bioprocess partly utilizes the fed CO₂ and release some of the unutilized CO₂ as a by-product. In this regard, the by-product CO₂ could be recycled back to the bioprocess to make the aforementioned integrated process more efficient. Optionally, a recycle unit, communicably coupled to the bioreactor and the compressor, is configured to recycle the by-product CO₂ back to the absorption chamber, via the compressor and the pre-filter.

Optionally, the bioprocess comprises an outlet for harvesting the grown microbial biomass from the bioreactor.

### DETAILED DESCRIPTION OF THE DRAWINGS

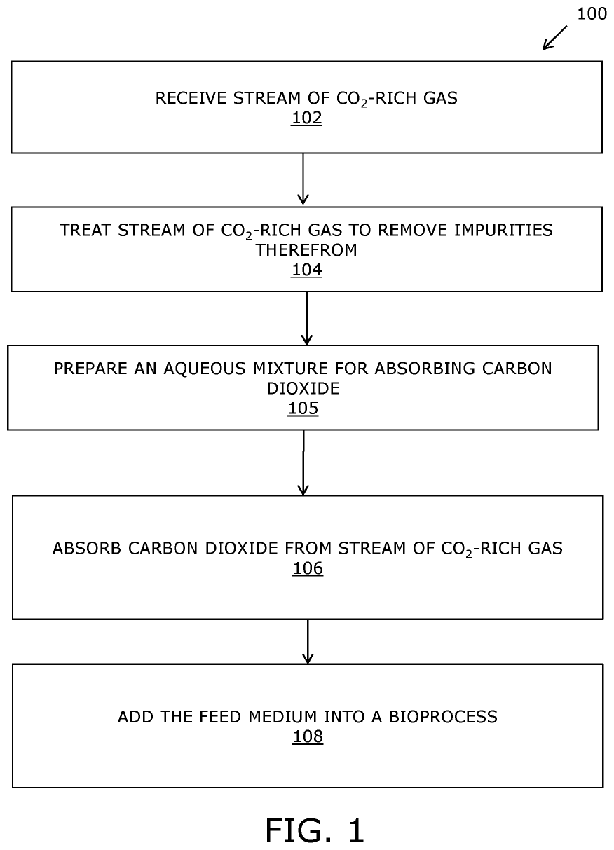

Referring to FIG. 1, illustrated is a flowchart 100 depicting steps of a method of adding a feed medium into a bioprocess, in accordance with an embodiment of the present disclosure. At step 102, a stream of CO₂-rich gas is received. At step 104, the stream of CO₂-rich gas is treated to remove impurities therefrom. At step 105, an aqueous mixture for absorbing carbon dioxide is prepared. At step 106, carbon dioxide from the stream of CO₂-rich gas is absorbed into an aqueous mixture, the aqueous mixture with absorbed carbon dioxide forming a feed medium. At step 108, the feed medium is added into a bioprocess.

The steps 102, 104, 105, 106 and 108 are only illustrative and other alternatives can also be provided where one or more steps are added, one or more steps are removed, or one or more steps are provided in a different sequence without departing from the scope of the claims herein.

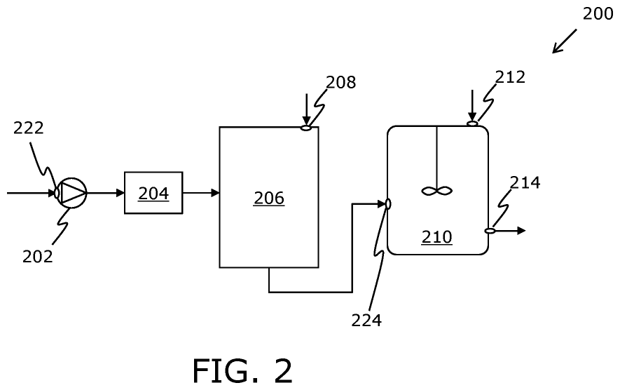

Referring to FIG. 2, there is shown a schematic illustration of a system 200 for adding a feed medium into a bioprocess, in accordance with an embodiment of the present disclosure. The system 200 comprises a first inlet 222 for providing a stream of CO₂-rich gas. Herein, the stream of CO₂-rich gas is provided to a pre-filter 204 via a compressor 202 for compressing the stream of CO₂-rich gas. The pre-filter 204 treats the stream of CO₂-rich gas to remove impurities therefrom. The system 200 comprises an absorption chamber 206 for absorbing carbon dioxide from the stream of CO₂-rich gas into an aqueous mixture. The absorbed carbon dioxide is coupled with a second inlet 208 for receiving an aqueous mixture, wherein the aqueous mixture with the absorbed carbon dioxide to form feed medium. The system 200 further comprises a third inlet 224 for adding the feed medium into a bioprocess. The feed medium is added to a bioreactor 210 that facilitates the bioprocess. The system 200 further comprises at least one fourth inlet 212 for adding at least one of: hydrogen gas, oxygen gas, carbon monoxide, minerals into the bioprocess; and a light source coupled to the bioreactor for illumination thereof. Moreover, the system 200 further comprises an outlet 214 for harvesting the grown microbial biomass from the bioreactor 210.

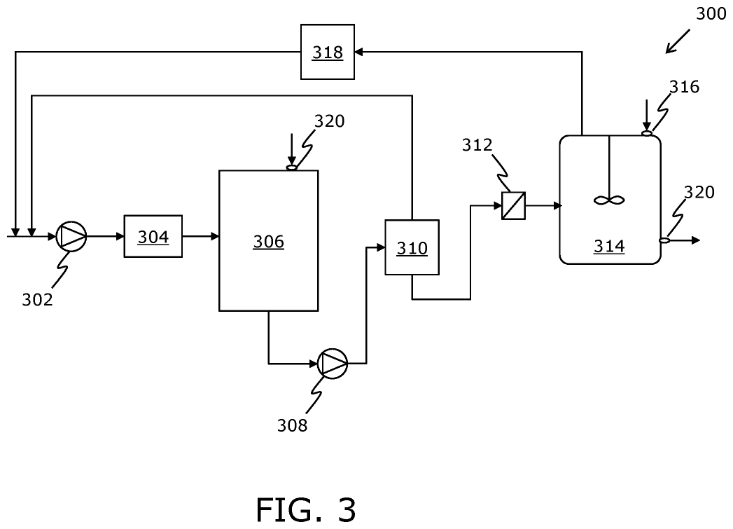

Referring to FIG. 3, there is shown a schematic illustration of a system 300 for adding a feed medium into a bioprocess, in accordance with an embodiment of the present disclosure. The system 300 comprises a pre-filter 304, wherein the stream of CO₂-rich gas is provided to a pre-filter 304 via a compressor 302. The system 300 comprises an absorption chamber 306 for absorbing carbon dioxide from the stream of CO₂-rich gas. The absorbed carbon dioxide is coupled with a second inlet 320 for receiving an aqueous mixture, wherein the aqueous mixture with the absorbed carbon dioxide to form feed medium. The system 300 further comprises a recycle unit 310, communicably coupled to the absorption chamber 306 and the filter 312. The recycle unit 310 is configured to recycle the carbon dioxide, water and one or more plurality of insoluble gases, received via a pump 308 after absorption of carbon dioxide back to the absorption chamber 306, via the compressor 302 and the pre-filter 304. As shown, the system 300 comprises a filter 312 for filtering the feed medium by removing impurities selected from plurality of solid impurities. Herein, the filter 312 is a sterile filter. The filtered feed medium from the filter 312 is provided to the bioreactor 314. The system 300 further comprises at least one fourth inlet 316 for adding at least one of: hydrogen gas, oxygen gas, carbon monoxide, minerals into the bioprocess; and a light source coupled to the bioreactor for illumination thereof. Moreover, the system 300 further comprises a recycle unit 318, communicably coupled to the bioreactor 314 and the compressor 302. The recycle unit 318 is configured to recycle the carbon dioxide, received via the bioreactor 314 as a by-product, back to the absorption chamber 306, via the compressor 302 and the pre-filter 304. Moreover, the system 300 further comprises an outlet 320 for harvesting the grown microbial biomass from the bioreactor 314.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A method of adding a feed medium into a bioprocess, the method comprising:
(a) receiving a stream of CO2-rich gas;
(b) treating the stream of CO2-rich gas to remove impurities therefrom;
(c) preparing an aqueous mixture for absorbing carbon dioxide, the aqueous mixture comprises at least one inorganic nitrogen compound in a range of 0.1-50 wt % of the aqueous mixture, the at least one inorganic nitrogen compound is a nitrogen source for microorganisms;
(d) absorbing carbon dioxide from the stream of CO₂-rich gas into the aqueous mixture, the aqueous mixture with absorbed carbon dioxide forming a feed medium; and
(e) adding the feed medium into a bioprocess.

2. The method according to claim 1, wherein the absorption of carbon dioxide is carried out at temperature ranging from 0 to 35° C. and pressure ranging from 1 to 200 bars.

3. The method according to claim 1 further comprising filtering the feed medium to remove impurities selected from plurality of solid impurities.

4. The method according to claim 1, wherein filtering is sterile filtering.

5. The method according to claim 1, further comprising adding at least one of: hydrogen gas, oxygen gas, carbon monoxide, minerals, light into the bioprocess.

6. The method according to claim 1, wherein the CO₂-rich gas is obtained from an external source, and wherein the external source is a combustion plant.

7. The method according to claim 6, wherein the external source further comprises microbial fermentation process to obtain CO₂-rich gases.

8. The method according to claim 1, further comprising recycling a recycled gas stream back to step (a), wherein the recycled gas stream comprises at least one of selected from the carbon dioxide, water and one or more plurality of insoluble gases generated at step (d) or the carbon dioxide generated at step (e), and wherein the stream of CO₂-rich gas is supplemented by the recycled gas stream.

9. The method according to claim 8, wherein the CO₂ concentration of the total volume of the stream of CO₂-rich gas supplemented by the recycled gas stream is determined by equation:
$$\\ X = \frac{( A \times B + A \times C )}{B + C} ,$$
wherein
X is concentration of CO₂ in the total volume of the stream of CO₂-rich gas supplemented by the recycled gas stream,
A is concentration of CO₂ in the stream of CO₂-rich gas,
B is flow rate of the stream of CO₂-rich gas,
C is flow rate of the recycled gas stream, and
D is concentration of CO₂ in the recycled gas stream.

10. The method according to claim 1, wherein concentration of the at least one inorganic nitrogen compound is in a range of 5-10 wt % of the aqueous mixture.

11. The method according to claim 1, wherein treating the CO2-rich gas comprises at least one of selected from filtering; pre-scrubbing; using a flash tank; desulphurisation; removal of hydrocarbons, oxygen, halogen, siloxanes;
filtering as high-efficiency particulate absorbing filtering.

12. A system for adding a feed medium into a bioprocess, the system comprising:
a first inlet for providing a stream of CO2-rich gas;
a pre-filter for treating the stream of CO2-rich gas to remove impurities therefrom;
an absorption chamber for absorbing carbon dioxide from the stream of CO₂-rich gas, and a second inlet for receiving an aqueous mixture that absorbs the carbon dioxide to form a feed medium, the aqueous mixture comprises at least one inorganic nitrogen compound in a range of 0.1-50 wt % of the aqueous mixture, the at least one inorganic nitrogen compound is a nitrogen source for microorganisms;
a third inlet for adding the feed medium into a bioprocess; and
a bioreactor (210, 314) for facilitating the bioprocess.

13. The system according to claim 12, further comprising a filter for filtering the feed medium by removing impurities selected from plurality of solid impurities.

14. The system according to claim 12, wherein the filter is a sterile filter.

15. The system according to claim 12, further comprising:
at least one fourth inlet for adding at least one of: hydrogen gas, oxygen gas, carbon monoxide, minerals into the bioprocess; and
a light source coupled to the bioreactor for illumination thereof.

16. The system according to claim 12, wherein the CO₂-rich gas is obtained from an external source, and wherein the external source is a combustion plant.

17. The system according claim 12, wherein the external source further comprises microbial fermentation process to obtain CO₂-rich gases.

18. The system according claim 12, wherein the bioreactor is configured for cultivating microorganisms selected from a group comprising autotrophic microorganisms, heterotrophic microorganisms, mixotrophic microorganisms, aerobic microorganisms, anaerobic microorganisms or facultative anaerobic microorganisms.

19. The system according to claim 12, further comprising at least one recycle unit, communicably coupled to the absorption chamber or bioreactor and the filter, configured to recycle the carbon dioxide, water and one or more plurality of insoluble gases generated in absorption chamber or in the bioreactor back to the first inlet of the aforementioned method.

20. The system according to claim 19 further comprising at least one sensor element configured to measure at least one of selected from the concentration of carbon dioxide, water and one or more plurality of insoluble gases generated in the adsorption chamber or the carbon dioxide generated in the bioreactor for determining the concentration of CO₂ necessary at the first inlet.

21. The system according to claim 12, wherein concentration of the at least one inorganic nitrogen compound is in a range of 5-10 wt % of the aqueous mixture

22. The system according to claim 12, further comprising at least one of selected from a pre-scrubber, a flash tank, an adsorber, a micro-aerator, a high-efficiency particulate absorbing filter.
